(19) United States
(12) Patent Application Publication
(10) Pub. No.: US 2021/0161840 A1
(43) Pub. Date: Jun. 3, 2021

(54) COMPOSITIONS AND METHODS FOR TREATMENT OF IRON OVERLOAD

(71) Applicant: Mordechai CHEVION, Mevaseret Zion (IL)

(72) Inventors: Mordechai CHEVION, Jerusalem (IL); Vladimir VINOKUR, Beer Sheva (IL)

(21) Appl. No.: 17/047,560

(22) PCT Filed: Apr. 11, 2019
(86) PCT No.: PCT/IL2019/050413

§ 371 Date: Oct. 14, 2020

Related U.S. Application Data
(60) Provisional application No. 62/657,821, filed on Apr. 15, 2018.

(51) Int. Cl. A61K 31/185; A61K 33/30; A61K 33/24; A61K 31/12; A61K 31/351; A61K 31/353; A61K 31/192; A61K 31/7048; A61K 31/4965; A61K 31/47; A61K 31/444; A61K 31/4412; A61K 31/4196; A61P 39/04; A61P 13/12

(52) U.S. Cl. CPC A61K 31/185; A61P 13/12; A61K 33/24; A61K 31/12; A61K 31/351; A61K 31/353; A61K 31/192; A61K 31/7048; A61K 31/4965; A61K 31/47; A61K 31/444; A61K 31/4412; A61K 31/4196; A61P 39/04; A61K 33/30

(57) ABSTRACT

The present invention relates to an iron chelator combination, more specifically a combination of a non-iron metal-desferrioxamine B complex and an additional iron chelator, for preventing, inhibiting, reducing or ameliorating iron overload or elevated levels of labile iron.

### TECHNICAL FIELD

The present invention relates to methods and compositions for preventing, inhibiting, reducing or ameliorating iron overload, thereby more particularly treating diseases, disorders, and conditions characterized by or associated with iron overload or excessive levels of labile and redox-active iron in tissues.

### BACKGROUND ART

Iron, a metallo-element abundant in mammalian tissues, including the human body, is an essential element for life, playing key roles in a variety of biological systems. In healthy adults, the total amount of iron is 3-4 g, of which about 1% is bound to iron-containing enzymes and redox-active proteins, including proteins involved in cellular respiration and electron transport.

“Labile iron pool” (LIP) is a small fraction of the total amount of iron. The LIP consists of labile and redox-active iron, which serves essential cellular purposes as well as the catalysis of production of reactive oxygen-derived species (ROS), including free radicals such as the hydroxyl radicals. ROS are known to generate oxidative stress, and to cause tissue injury and inflammation.

Severe iron overload has been recognized as highly toxic for about two centuries. The most common causes of chronic iron overload are hereditary, transfusional, and acquired disorders including hereditary hemochromatosis (HHC) and thalassemia. Only at the late 1970s it became evident that LIP, even under normal iron status, can cause cellular and tissue injury, leading to a broad spectrum of pathologies.

These pathological conditions involve imbalance of the levels of transition metals and include, e.g., pantothenate kinase-associated neurodegeneration, human immunodeficiency virus infection and acquired immune deficiency syndrome, intracerebral hemorrhage, myelodysplastic syndrome, Hodgkin lymphoma, non-Hodgkin lymphoma, hepatic insufficiency, renal failure, sickle-cell disease, Parkinson's disease, Friedreich's ataxia, thalassemia, amyotrophic lateral sclerosis, neurodegeneration with brain iron accumulation, superficial siderosis, contrast-induced acute kidney injury, iron overload due to stem cell transplant, mucormycosis, acute myeloid leukemia, Diamond-Blackfan anemia, hemolytic anemia, porphyria cutanea tarda, malaria, acute lymphoid leukemia, hemosiderosis, non-alcoholic steatohepatitis, aplastic anemia, diabetic nephropathy, glomerulonephritis, rheumatoid arthritis, endotoxemia, stroke, chronic kidney disease, systemic sclerosis, Wilson's disease, Menkes disease, glioblastoma, pulmonary fibrosis, idiopathic pulmonary fibrosis, chronic hemophilic synovitis, Alzheimer's disease, Huntington's disease, schizophrenia, cystinuria, biliary cirrhosis, leishmaniasis, multiple sclerosis, cholangiocarcinoma, primary sclerosing cholangitis, heavy metals poisoning, autoimmune encephalomyelitis, carcinoma, fibrosarcoma, fibroma, histiocytoma, myxosarcoma, angiomyxoma, adenoma, mesothelioma, hepatoblastoma, adenocarcinoma, cholangiocarcinoma, cystadenoma, melanoma, sarcoma, hemangioma, teratoma, adenomyoma, leiomyosarcoma, oncocytoma, inverted papilloma, papilloma, inflammatory bowel disease, ulcerative colitis, Crohn's disease, diabetes mellitus, and psoriasis. Furthermore, excessive accumulation of labile redox-active iron in heart was reported to underlie the cardiotoxic effects of some chemotherapy drugs (Gammella et al., 2014).

Routine treatment of iron overload in an otherwise-healthy person consists of regularly scheduled phlebotomies. Patients unable to tolerate routine blood draws can use medications that act as iron chelating agents.

The most widely used iron chelating drug is Desferal®, which is the mesylate salt of desferrioxamine B (DFO). DFO is a siderophore, i.e., a small molecule with high-affinity for ferric iron, which is secreted by microorganisms and serves as a scavenger for environmental iron and as a shuttle for the importation of iron into the microbial cells. DFO is synthesized by the generally recognized as safe (GRAS) actinobacteria Streptomyces pilosus. Desferal® was developed by Ciba Geigy as a medication for clearance of iron overload, and was approved by the FDA for clinical use in 1964. Due to the large amounts of iron deposited within different tissues of hemochromatotic patients, and the low solubility of Desferal® in lipid phase, daily doses of >4000 mg/day/person were and still are being administered in patients. Structurally, DFO is a long, linear, hydrophilic molecule, which slowly and sparingly penetrates cell membranes, and barely enters tissues. Therefore, routes of Desferal® administration are limited to intramuscular, subcutaneous, and intravenous injections only.

To overcome the limitations of the clinical use of Desferal®, described above, ‘non-iron’ metal-ion complexes of DFO, such as zinc and gallium complexes of DFO, were prepared (U.S. Pat. Nos. 5,075,469 and 5,618,838). These complexes were found to be more effective than Desferal® alone in treatment of iron-mediated cell and tissue injury. In another approach to overcome these limitations, Desferal® is administered together with another iron chelating agent having a lower affinity to iron, preferably a cell-permeable one (Hider, 2010), such as deferiprone, which readily penetrates cellular membrane and can be administered also orally. The daily doses of both chelators used have exceeded 30 mg/kg (Origa et al., 2005).

Presumably, being administered in such a combination, deferiprone acts as a shuttle, exporting iron from various intracellular compartments to the outside of the cell and there transferring the iron to the DFO. Yet, long-term administration of iron chelators are frequently accompanied by various adverse side effects of different severity, including intestinal bleeding or sores at the site of the injection.

The spatial structures of Zn-DFO and Ga-DFO complexes are markedly different from that of DFO alone, and characterized by more compact structures, where the DFO is coiled around the metal ion, yielding improved capacity of the complex to infiltrate into cells and tissues (Chevion, 1998 and 1991), which allows the scavenging of the LIP through an exchange of the DFO-bound zinc (or gallium) ion by intracellular ferric iron ion. Zn-DFO or Ga-DFO complex can be administered in a wide range of concentrations, typically 2-6 mg/kg that is noticeably lower than DFO in its metal-free form. As already shown, administration of the Zn-DFO complex in amounts of 2-6 mg/kg provided protection against the development of asthma in the mouse/ovalbumin model of human asthma (Bibi et al., 2014).

### SUMMARY OF INVENTION

In one aspect, the present invention relates to a method for preventing, inhibiting, reducing or ameliorating iron overload or elevated levels of labile (and thus redox-active) iron in a subject in need thereof, thereby more specifically treating a disease, disorder or condition characterized by or associated with iron overload or elevated levels of labile iron, said method comprising administering to said subject a therapeutically effective amount of a combination comprising a metal-desferrioxamine B complex (metal-DFO complex) or a pharmaceutically acceptable salt thereof, wherein said metal is not iron, and an additional iron chelator.

In another aspect, the present invention provides a pharmaceutical composition comprising a combination of a metal-DFO complex or a pharmaceutically acceptable salt thereof, wherein said metal is not iron, and an additional iron chelator, and a pharmaceutically acceptable carrier. Such a pharmaceutical composition is useful in preventing, inhibiting, reducing or ameliorating iron overload or elevated levels of labile iron, thereby more specifically treating a disease, disorder or condition characterized by or associated with iron overload or elevated levels of labile iron.

In still another aspect, the present invention relates to a combination of a metal-DFO complex or a pharmaceutically acceptable salt thereof, wherein said metal is not iron, and an additional iron chelator, for use in preventing, inhibiting, reducing, or ameliorating iron overload or elevated levels of labile iron.

In yet another aspect, the present invention relates to use of a combination of a metal-DFO complex or a pharmaceutically acceptable salt thereof, wherein said metal is not iron, and an additional iron chelator in the preparation of a pharmaceutical composition for preventing, inhibiting, reducing, or ameliorating iron overload or elevated levels of labile iron.

In a further aspect, the present invention provides a kit comprising:
(i) either a pharmaceutical composition A comprising a metal-DFO complex or a pharmaceutically acceptable salt thereof; or pharmaceutical compositions B and C, wherein pharmaceutical composition B comprises DFO or a pharmaceutically acceptable salt thereof, and pharmaceutical composition C comprises ions of a metal, wherein said metal is not iron;
(ii) a pharmaceutical composition D comprising an additional iron chelator; and
(iii) instructions to administer either (a) pharmaceutical compositions A and D, either concomitantly or sequentially at any order and within a time period not exceeding 36 hours; or (b) pharmaceutical compositions B, C and D, either concomitantly or sequentially at any order and within a time period not exceeding 36 hours, so as to form in situ, upon complexation of said DFO or pharmaceutically acceptable salt thereof and said metal ions, a metal-DFO complex or a pharmaceutically acceptable salt thereof, to thereby prevent, inhibit, reduce, or ameliorate iron overload or elevated levels of labile iron.

### DETAILED DESCRIPTION OF THE INVENTION

The term “DFO”, “deferoxamine” or “desferrioxamine B”, used herein interchangeably, refers to the compound N′-[5-(acetyl-hydroxy-amino)pentyl]-N-[5-[3-(5-aminopentyl-hydroxy-carbamoyl)propanoylamino]pentyl]-N-hydroxy-butane diamide, a bacterial siderophore, which is made up from six basic units and naturally produced by the actinobacteria Streptomyces pilosus. When not bound to a metal, DFO is a linear, noodle-like, molecule that sparingly infiltrates into cells; however, upon metal binding it becomes less polar and assumes a globular complex capable of infiltrating into cells. These considerations explain why the DFO complexes more easily penetrate through cellular membranes, and more effectively bind intracellular iron that is redox active and mediates tissue damage.

It is postulated that some of the useful effects exerted by DFO in inhibiting ROS formation are achieved through its actions as a chelating agent of ferric iron (chelant, chelator, or sequestering agent). In addition, DFO is capable of forming soluble complexes, i.e., chelates, with certain (non-iron) metal ions, that are redox-inactive and consequently they cannot normally react with other elements or ions. Such chelates often have chemical and biological properties that are markedly different from those of either the chelator or the metal ion, alone.

In addition to iron, DFO forms a tight complex with redox-silent metals such as zinc and gallium. In recent experiments comparing the ability of DFO alone and the Zn-DFO complex to infiltrate into cells in a tissue culture model using H9C2 cardiomyocytes, it has been found that the Zn-DFO complex infiltrates into the cells more than three-fold faster than DFO alone (data not shown). Using the zinc (or a different non-iron metal) complex of DFO may thus provide two-step antioxidant protection, wherein the redox-active iron is chelated and its redox activity is arrested; and the zinc that had been a part of the DFO complex, which in itself possesses antioxidant activity and is needed for the adequate functioning of various enzymes, or the other non-iron metal, is then released in a controlled manner.

Desferal® is a commercially available DFO marketed in the form of its methanesulfonate (mesylate) salt. Other pharmaceutically acceptable salts of DFO include, without being limited to, the chloride, bromide, iodide, acetate, ethanesulfonate (esylate), ethanedisulfonate (edisylate), maleate, fumarate, tartrate, bitartrate, sulfate, p-toluenesulfonate, benzenesulfonate, tosylate, benzoate, acetate, phosphate, carbonate, bicarbonate, succinate, and citrate salt thereof.

The relative stability constants for the DFO complexes with Fe(III), Cu(II), Zn(II) and Ga(III) are 10³¹, 10¹⁴, 10¹¹ and 10²⁸, respectively (Keberle, 1964). The stability constant of a DFO complex with a lanthanide ions is expected to be lower than 10³¹ (Orcutt et al., 2010). Based on these thermodynamic properties, upon penetration into cells, with high abundance of labile and redox-active Fe, the Zn-DFO complex exchanges the Zn with Fe, and the zinc released from the complex could have an additional beneficial antioxidant and/or other effects.

The therapeutical concept underlying the present invention is the use of a combination comprising, or consisting of, a non-iron metal-DFO complex (herein also referred to a “Zygosid”), e.g., Zn-DFO, Ga-DFO, or a mixture thereof, and an additional different iron chelator, preferably bound to a non-iron metal such as Zn or Ga, which will infiltrate into cells, and substantially more effectively reduce or ameliorate iron overload or elevated levels of labile iron. The efficacy of a combination of two or more chelates, each containing, e.g., zinc or gallium, where both chelates are located intracellularly, is expected to remarkably improve the scavenging efficacy of labile iron from various intracellular compatrents. The therapeutical concept of the present invention is thus expected to prove highly efficacious using significantly reduced doses of the chelators in the removal of excess iron from the body.

In one aspect, the present invention relates to a method for preventing, inhibiting, reducing or ameliorating iron overload or elevated levels of labile (and thus redox-active) iron, thereby more specifically treating a disease, disorder or condition characterized by or associated with iron overload or elevated levels of labile iron, in a subject in need thereof, said method comprising administering to said subject a therapeutically effective amount of a combination comprising a metal-DFO complex or a pharmaceutically acceptable salt thereof, wherein said metal is not iron (herein also referred to as a “non-iron metal-DFO complex”), and an additional iron chelator that may be either partly or fully saturated with a non-redox active metal ion.

In certain embodiments, the non-iron metal-DFO complex administered according to the method of the present invention is the zinc-DFO complex, gallium-DFO complex, manganese-DFO complex, copper-DFO complex, aluminum-DFO complex, vanadium-DFO complex, indium-DFO complex, chromium-DFO complex, gold-DFO complex, silver-DFO complex, or platinum-DFO complex, a lanthanide-DFO complex, or a mixture thereof. Partiuclar lanthanides include lanthanum, cerium, praseodymium, neodymium, promethium, samarium, europium, gadolinium, terbium, dysprosium, holmium, erbium, thulium, ytterbium, and lutetium, of which europium and gadolinium are preferred. According to the invention, in cases wherein a mixture of two or more metal-DFO complexes is administered, said mixture may comprise said metal-DFO complexes in any quantitative ratio. For example, in case a mixture of two metal-DFO complexes is administered, said mixture may comprise said two metal-DFO complexes in a quantitative ratio of about 100:1 to about 1:100, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100. Similarly, in case a mixture of three metal-DFO complexes is administered, said mixture may comprise said three metal-DFO complexes in a quantitative ratio of, e.g., about 1:1:1, about 1:2:3, about 1:10:50, about 1:20:50, about 1:10:100, or about 1:50:100.

In particular embodiments, the non-iron metal-DFO complex administered according to the method of the present invention is Zn-DFO complex, Ga-DFO complex, or a mixture of Zn-DFO complex and any one of the other non-iron metal-DFO complexes listed above, e.g., Ga-DFO complex. In more particular such embodiments, a mixture of Zn-DFO complex and an additional metal-DFO complex, e.g., Ga-DFO complex, is administered, e.g., wherein the quantitative ratio of the Zn-DFO complex to the other metal-DFO complex is in a range of about 100:1 to about 1:100, e.g., about 50:1 to about 1:50, about 40:1 to about 1:40, about 30:1 to about 1:30, about 20:1 to about 1:20, about 10:1 to about 1:10, about 5:1 to about 1:5, about 4:1 to about 1:4, about 3:1 to about 1:3, about 2:1 to about 1:2, or about 1:1. Certain such mixtures are those wherein the amount of the Zn-DFO complex is higher than that of the other metal-DFO complex, e.g., mixtures wherein the quantitative ratio of the Zn-DFO complex to the other metal-DFO complex is in a range of about 10:1 to about 2:1, e.g., about 10:1, about 9:1, about 8:1, about 7:1, about 6:1, about 5:1, about 4:1, about 3:1, or about 2:1. Other such mixtures are those wherein the amount of the Zn-DFO complex is lower than that of the other metal-DFO complex, e.g., mixtures wherein the quantitative ratio of the Zn-DFO complex to the other metal-DFO complex is in a range of about 1:2 to about 1:10, e.g., about 1:2, about 1:3, about 1:4, about 1:5, about 1:6, about 1:7, about 1:8, about 1:9, or about 1:10.

The term “iron chelator” or “iron chelating agent/molecule” denotes a molecule capable of chelating iron, i.e., forming coordinate bonds with iron ion, and the term “iron chelation therapy” refers to the removal of excess iron from the body by administration of an iron chelator. The term “additional iron chelator” as used herein denotes an iron chelating molecule other than DFO, a non-iron metal complex thereof, or a pharmaceutically acceptable salt thereof.

In certain embodiments, the additional iron chelator administered according to the method of the present invention is a natural siderophore, i.e., a small, high-affinity iron-chelating compound naturally produced and secreted by a microorganism such as bacteria or fungi and serving to transport iron across cell membrane, or a derivative thereof, i.e., a chemically modified siderophore. Examples of natural siderophores include, without being limited to, desferrioxamine D (DFO D), and desferrioxamine E (DFO E; nocardamine); and examples of chemically modified siderophores include, without being limited to, desferrioxamine B analogs such as those disclosed in Kornreich-Leshem, 2003 and 2005), herewith incorporated by reference in their entirety as if fully disclosed herein.

In other embodiments, the additional iron chelator administered according to the method of the present invention is desferrithiocin; kojic acid; 2,3-, 2,4-, 2,5-, 2,6-, 3,4-, or 3,5-dihydroxybenzoic acid (DHBA); a synthetic chelator such as deferiprone, ethylenediaminetetraacetic acid (EDTA), clioquinol, dimercaptosuccinic acid (DMSA), O-trensox, deferasirox, dexrazoxane, tachpyr, triapine, dimercaprol, penicillamine, pyridoxal isonicotinoyl hydrazone (PIH), and di-2-pyridylketone thiosemicarbazone; a phytochemical such as genistein, phytic acid, theaflavin, epigallochatechin gallate (EGCG), quercetin, ligustrazine, baicalin, baicalein, floranol, kolaviron, apocynin, flavan-3-ol, and curcumin; or a pharmaceutically acceptable salt of the aforesaid.

The additional iron chelator administered according to the method of the present invention may be either in a metal-free form, or in a complex with a non-iron metal such as zinc, gallium, manganese, copper, aluminum, vanadium, indium, chromium, gold, silver, platinum, or a lanthanide such as lanthanum, cerium, praseodymium, neodymium, promethium, samarium, europium, gadolinium, terbium, dysprosium, holmium, erbium, thulium, ytterbium, and lutetium.

According to the method of the present invention, the non-iron metal-DFO complex and additional iron chelator administered can be at any quantitative ratio. In certain embodiments, the quantitative ratio of said metal-DFO complex to said additional iron chelator in said combination is in a range of 100:1 to 1:100, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100.

In certain embodiments, the non-iron metal-DFO complex and additional iron chelator administered according to the method of the present invention, as defined in any one of the embodiments above, are formulated as separate, e.g., two, pharmaceutical compositions for administration either concomitantly or sequentially at any order and within a time period not exceeding 36 hours, through one or more routes of administration. According to the method of the invention, each one of the compositions administered may be independently formulated for any suitable administration route, e.g., for oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

In other embodiments, the non-iron metal-DFO complex and additional iron chelator administered according to the method of the present invention, as defined in any one of the embodiments above, are formulated as a sole pharmaceutical composition. Such a composition may be formulated for any suitable administration route, e.g., for oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

The method disclosed herein, according to any one of the embodiments defined above, is aimed at preventing, inhibiting, reducing or ameliorating iron overload or elevated levels of labile iron, thereby treating a disease, disorder or condition characterized by or associated with iron overload or elevated levels of labile iron in a subject in need thereof, by administering a therapeutically effective amount of an iron chelator combination, also referred to herein as active agent/drug combination, comprising a non-iron metal-DFO complex or a pharmaceutically acceptable salt thereof, and an additional iron chelator optionally partly or fully saturated with a non-redox active metal ion.

The term “iron overload” is also known as hemochromatosis or haemochromatosis, and indicates accumulation of iron in the body tissues, from any cause. The most important causes are hereditary haemochromatosis, a genetic disorder, and transfusional iron overload that may result from repeated blood transfusion.

Haemochromatosis may be either primary, i.e., hereditary or genetically determined, or secondary that is less frequent and acquired during life. While the majority of primary haemochromatosis depends on mutations of the human hemochromatosis protein (HFE) gene, other cases of primary haemochromatosis result from mutations in other genes and further referred to as “non-classical hereditary haemochromatosis”, “non-HFE related hereditary haemochromatosis”, or “non-HFE haemochromatosis”. Secondary haemochromatosis may result from various medical conditions such as severe chronic haemolysis of any cause, including intravascular haemolysis and ineffective erythropoiesis; multiple frequent blood (either whole blood or red blood cells) transfusions required by individuals with hereditary anaemias (such as beta-thalassaemia major, sickle cell anaemia, and Diamond-Blackfan anaemia) or by older patients suffering from severe acquired anaemias such as in myelodysplastic syndromes; excess parenteral iron supplements (e.g., iron poisoning); and excess dietary iron. The term “elevated (or excess) levels of labile iron”, also known as elevated levels of LIP, denotes an excess amout of the small fraction of the total tissue iron, which is non-protein bound (sometimes incorrectly referred to as “free iron”), and is labile and redox-active, thus serving as a catalyst for the production of ROS, yielding pathologic proesses.

The term “subject” as used herein refers to any mammal, e.g., a human, non-human primate, horse, ferret, dog, cat, cow, and goat. In a preferred embodiment, the term “subject” denotes a human, i.e., an individual.

The term “treatment” as used herein with respect to a disease, disorder or condition characterized by or associated with iron overload or elevated levels of labile iron, refers to the administration of a therapeutically effective amount of an iron chelator combination as described above, which is effective to ameliorate undesired symptoms associated with said disease, disorder or condition; prevent the manifestation of such symptoms before they occur; slow down the progression of said disease, disorder or condition; slow down the deterioration of symptoms; enhance the onset of remission period; slow down the irreversible damage caused in the progressive chronic stage of said disease, disorder or condition; delay the onset of said progressive stage; lessen the severity or cure said disease, disorder or condition; improve survival rate or more rapid recovery; and/or prevent said disease, disorder or condition form occurring.

The term “therapeutically effective amount” as used herein with respect to the drug combination administered according to the method of the invention refers to an amount of said drug combination, more partiucarly amounts of said metal-DFO complex and said additional iron chelator, that upon administration under a particular regimen during a particular period of time, e.g., days, weeks, months or years, is sufficient to prevent, inhibit, reduce or ameliorate an iron overload occurring in the body of the subject administered with. The actual dosages of both the metal-DFO complex and the additional iron chelator administered may be varied so as to obtain amounts of said metal-DFO complex and said additional iron chelator that are effective to achieve the desired prophylactic/therapeutic response for a particular subject and mode of administration, without being toxic to the subject. The dosage level selected will depend upon a variety of factors including the activity of the metal-DFO complex employed, the route of administration, the duration of the treatment, and other drugs, if any, used in addition to the drug combination employed, as well as the age, sex and weight of the subject treated, and the severity/progression of the medical condition. In general, it may be presumed that for preventive treatment, lower doses will be needed, while higher doses will be required for treatment of subjects already showing pathological phenotypes of said iron overload. It may further be presumed that the effective dose of an iron chelator combination will be lower than that required to achieve the same therapeutic end point (result) by using only one iron chelator of those constituting said combination.

In certain embodiments, the disease, disorder or condition characterized by or associated with iron overload or elevated levels of labile iron, and thus prevented, inhibited, reduced or ameliorated by the method of the present invention include, without limiting, pantothenate kinase-associated neurodegeneration, human immunodeficiency virus infection and acquired immune deficiency syndrome, intracerebral hemorrhage, myelodysplastic syndrome, Hodgkin lymphoma, non-Hodgkin lymphoma, hepatic insufficiency, renal failure, sickle-cell disease, Parkinson's disease, Friedreich's ataxia, thalassemia, amyotrophic lateral sclerosis, neurodegeneration with brain iron accumulation, superficial siderosis, contrast-induced acute kidney injury, iron overload due to stem cell transplant, mucormycosis, acute myeloid leukemia, Diamond-Blackfan anemia, hemolytic anemia, porphyria cutanea tarda, malaria, acute lymphoid leukemia, hemosiderosis, non-alcoholic steatohepatitis, aplastic anemia, diabetic nephropathy, glomerulonephritis, rheumatoid arthritis, endotoxemia, stroke, chronic kidney disease, systemic sclerosis, Wilson's disease, Menkes disease, glioblastoma, pulmonary fibrosis, idiopathic pulmonary fibrosis, chronic hemophilic synovitis, Alzheimer's disease, Huntington's disease, schizophrenia, cystinuria, biliary cirrhosis, leishmaniasis, multiple sclerosis, cholangiocarcinoma, primary sclerosing cholangitis, heavy metals poisoning, autoimmune encephalomyelitis, carcinoma, fibrosarcoma, fibroma, histiocytoma, myxosarcoma, angiomyxoma, adenoma, mesothelioma, hepatoblastoma, adenocarcinoma, cholangiocarcinoma, cystadenoma, melanoma, sarcoma, hemangioma, teratoma, adenomyoma, leiomyosarcoma, oncocytoma, inverted papilloma, papilloma, chemotherapy-induced iron overload, inflammatory bowel disease, ulcerative colitis, Crohn's disease, diabetes mellitus, metabolic syndrome, and psoriasis.

In other embodiments, the disorder or condition characterized by or associated with iron overload or elevated levels of labile iron, and thus prevented, inhibited, reduced or ameliorated by the method of the present invention is induced by an injury, such as an injury caused by a mechanical force, ischemia, a toxic agent such as an herbicide or pesticide, or hemorrhage.

In another aspect, the present invention provides a pharmaceutical composition comprising a drug combination as defined above, i.e., a combination of a non-iron metal-DFO complex or a pharmaceutically acceptable salt thereof, and an additional iron chelator optionally partly or fully saturated with a non-redox active metal ion, and a pharmaceutically acceptable carrier.

The drug combination comprised within the pharmaceutical composition of the present invention may be any combination of a non-iron metal-DFO complex or a pharmaceutically acceptable salt thereof, and an additional iron chelator.

In certain embodiments, the non-iron metal-DFO complex comprised within the pharmaceutical composition of the invention is the zinc-DFO complex, gallium-DFO complex, manganese-DFO complex, copper-DFO complex, aluminum-DFO complex, vanadium-DFO complex, indium-DFO complex, chromium-DFO complex, gold-DFO complex, silver-DFO complex, or platinum-DFO complex, a lanthanide-DFO complex, or a mixture thereof. Mixtures of metal-DFO complexes, when used, may comprise two metal-DFO complexes in any quantitative ratio, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100. Other mixtures may comprise three metal-DFO complexes in any quantitative ratio, e.g., in a quantitative ratio of, e.g., about 1:1:1, about 1:2:3, about 1:10:50, about 1:20:50, about 1:10:100, or about 1:50:100.

In particular embodiments, the metal-DFO complex comprised within the pharmaceutical composition of the the invention is Zn-DFO complex, Ga-DFO complex, or a mixture of Zn-DFO complex and any one of the other non-iron metal-DFO complexes listed above, e.g., Ga-DFO complex. In more particular such embodiments, a mixture of Zn-DFO complex and an additional metal-DFO complex, e.g., Ga-DFO complex, is used, e.g., wherein the quantitative ratio of the Zn-DFO complex to the other metal-DFO complex is in a range of about 100:1 to about 1:100, e.g., about 50:1 to about 1:50, about 40:1 to about 1:40, about 30:1 to about 1:30, about 20:1 to about 1:20, about 10:1 to about 1:10, about 5:1 to about 1:5, about 4:1 to about 1:4, about 3:1 to about 1:3, about 2:1 to about 1:2, or about 1:1. Certain such mixtures are those wherein the amount of the Zn-DFO complex is higher than that of the other metal-DFO complex, e.g., mixtures wherein the quantitative ratio of the Zn-DFO complex to the other metal-DFO complex is in a range of about 10:1 to about 2:1, e.g., about 10:1, about 9:1, about 8:1, about 7:1, about 6:1, about 5:1, about 4:1, about 3:1, or about 2:1. Other such mixtures are those wherein the amount of the Zn-DFO complex is lower than that of the other metal-DFO complex, e.g., mixtures wherein the quantitative ratio of the Zn-DFO complex to the other metal-DFO complex is in a range of about 1:2 to about 1:10, e.g., about 1:2, about 1:3, about 1:4, about 1:5, about 1:6, about 1:7, about 1:8, about 1:9, or about 1:10.

In certain embodiments, the additional iron chelator comprised within the pharmaceutical composition of the invention is a natural siderophore or a derivative thereof. Examples of natural siderophores and derivatives thereof are listed above. In other embodiments, the additional iron chelator comprised within the pharmaceutical composition of the invention is desferrithiocin; kojic acid; DHBA; a synthetic chelator such as deferiprone, EDTA, clioquinol, DMSA, O-trensox, deferasirox, dexrazoxane, tachpyr, triapine, dimercaprol, penicillamine, PIH, and di-2-pyridylketone thiosemicarbazone; a phytochemical such as genistein, phytic acid, theaflavin, EGCG, quercetin, ligustrazine, baicalin, baicalein, floranol, kolaviron, apocynin, flavan-3-ol, and curcumin; or a pharmaceutically acceptable salt of the aforesaid. The additional iron chelator comprised within the pharmaceutical composition of the invention may be either in a metal-free form, or in a complex with a metal such as zinc, gallium, manganese, copper, aluminum, vanadium, indium, chromium, gold, silver, platinum, or a lanthanide.

The drug combination comprised within the pharmaceutical composition of the present invention may contain the non-iron metal-DFO complex and the additional iron chelator at any quantitative ratio. In certain embodiments, the quantitative ratio of said metal-DFO complex to said additional iron chelator in the drug combination is in a range of 100:1 to 1:100, e.g., in a quantitative ratio of about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, or about 1:100.

The metal-DFO complexes for use according to the method and composition of the present invention may be prepared utilizing any technology or procedure known in the art, e.g., as described in International Publication No. WO2011021203. Possible procedures for the preparation of Zn-DFO and Ga-DFO complexes having particular metal:DFO stoichiometric ratios are provided herein below. Other such complexes having different metal:DFO stoichiometric ratios may be prepared using similar procedures.

A Zn-DFO complex having Zn:DFO stoichiometric ratio of 1.0:1.0 may be prepared, e.g., by mixing 10 mM solution of DFO with an equal volume of 10 mM ZnCl₂ solution, titrating to a pH between 5.0 to 7.5 , heating the mixture to 45° C. for 30 min, and cooling down. Alternatively, such a complex may be prepared by drying the contents of 1 vial (500 mg, 0.76 mmole) of Desferal®, by adding 168 mg of dry zinc acetate anhydrous (0.76 mmole), adding double distilled water until the contents fully dissolve (about 10 ml), warming the solution to 40° C. for 45 minutes, and cooling down.

A Zn-DFO complex having Zn:DFO stoichiometric ratio of 1.0:1.25 may be prepared, e.g., by mixing 10 mM solution of DFO with an equal volume of 6 mM ZnCl₂ solution, titrating to a pH between 5.0 to 7.5 , heating to 45° C. for 30 min, and cooling down.

A Zn-DFO complex having Zn:DFO stoichiometric ratio of 0.6:1.0 may be prepared, e.g., by mixing 10 mM DFO solution with an equal volume of 12.5 mM ZnCl₂ solution and 10 ml of 5.5 mM histidine, titrating to a pH between 5.0 to 7.5 , heating to 45° C. for 30 min, and cooling down.

A Zn-DFO complex having Zn:DFO stoichiometric ratio of 0.2:1.0 may be prepared, e.g., by mixing 50 mM DFO solution with ⅕ the volume of 50 mM ZnSO₄ solution, at the same pH recited above, heating to 40° C. for 45 min, and cooling down.

A Ga-DFO complex having Ga:DFO stoichiometric ratio of 1.0:1.0 may be prepared, e.g., by mixing 10 mM solution of DFO with an equal volume of 10 mM GaCl₃ solution, titrating to pH of about 5.0 and then to a pH between 6.0 to 7.5 (using NaOH). A similar complex having Ga:DFO stoichiometric ratio of 0.6:1.0 may be prepared, e.g., by mixing 5 mM DFO solution with an equal volume of 3 mM GaCl₃ solution, titrating to a pH between 5.0 to 7.5.

Pharmaceutical compositions as disclosed herein may be prepared by conventional techniques, e.g., as described in Remington: The Science and Practice of Pharmacy, 19^{th} Ed., 1995. The compositions can be prepared, e.g., by uniformly and intimately bringing the active agents, i.e., the non-iron metal-DFO complex(es) and the additional iron chelator, optionally partly or fully saturated with a non-redox active metal ion, into association with a liquid carrier, a finely divided solid carrier, or both, and then, if necessary, shaping the product into the desired formulation. The active agents may be applied as is, or conjugated to one or more pharmaceutically acceptable groups such as sugars, starches, amino acids, polyethylene-glycol (PEG), polyglycerol-based compounds, hydrazines, hydroxylamines, amines, or halides. The compositions may be in the form of a liquid (e.g., solution, emulsion, or suspension), gel, cream, solid, semisolid, film, foam, lyophilisate, or aerosol, and may further include pharmaceutically and physiologically acceptable fillers, carriers, diluents or adjuvants, and other inert ingredients and excipients. In one embodiment, the pharmaceutical composition of the invention is formulated as nanoparticles or microparticles.

The pharmaceutical compositions of the present invention may be formulated for any suitable route of administration, e.g., oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

The pharmaceutical compositions of the invention, when formulated for oral administration, may be in any suitable form, e.g., tablets, troches, lozenges, aqueous, or oily suspensions, dispersible powders or granules, emulsions, hard or soft capsules, or syrups or elixirs. In certain embodiments, said tablets are in the form of matrix tablets in which the release of a soluble active agent(s) is controlled by having the active agent(s) diffuse through a gel formed after the swelling of a hydrophilic polymer brought into contact with dissolving liquid (in vitro) or gastro-intestinal fluid (in vivo). Many polymers have been described as capable of forming such gel, e.g., derivatives of cellulose, in particular the cellulose ethers such as hydroxypropyl cellulose, hydroxymethyl cellulose, methylcellulose or methyl hydroxypropyl cellulose, and among the different commercial grades of these ethers are those showing fairly high viscosity. In other embodiments, the tablets are formulated as bi- or multi-layer tablets, made up of two or more distinct layers of granulation compressed together with the individual layers lying one on top of another, with each separate layer containing a different active agent. Bilayer tablets have the appearance of a sandwich since the edge of each layer or zone is exposed. In further embodiments, the compositions comprise the active agent(s) formulated for controlled release in microencapsulated dosage form, in which small droplets of the active agent(s) are surrounded by a coating or a membrane to form particles in the range of a few micrometers to a few millimeters.

Pharmaceutical compositions for oral administration might be formulated so as to inhibit the release of one or both of the active agents in the stomach, i.e., delay the release of one or both of the active agents until at least a portion of the dosage form has traversed the stomach, in order to avoid the acidity of the gastric contents from hydrolyzing the active agent. Particular such compositions are those wherein the active agent is coated by a pH-dependent enteric-coating polymer. Examples of pH-dependent enteric-coating polymer include, without being limited to, Eudragit® S (poly(methacrylicacid, methylmethacrylate), 1:2), Eudragit® L 55 (poly (methacrylic acid, ethylacrylate), 1:1), Kollicoat® (poly(methacrylicacid, ethylacrylate), 1:1), hydroxypropyl methylcellulose phthalate (HPMCP), alginates, carboxymethylcellulose, and combinations thereof. The pH-dependent enteric-coating polymer may be present in the composition in an amount from about 10% to about 95% by weight of the entire composition.

In certain embodiments, the invention provides a pharmaceutical composition for oral administration, which is solid and may be in the form of granulate, granules, grains, beads or pellets, mixed and filled into capsules or sachets, or compressed to tablets by conventional methods. In some particular embodiments, the pharmaceutical composition is in the form of a bi- or multilayer tablet, in which each one of the layers comprise one of the two active agents, and the layers are optionally separated by an intermediate, inactive layer, e.g., a layer comprising one or more disintegrants.

Another contemplated formulation is depot systems, based on biodegradable polymers. As the polymer degrades, the active agent(s) is slowly released. The most common class of biodegradable polymers is the hydrolytically labile polyesters prepared from lactic acid, glycolic acid, or combinations of these two molecules. Polymers prepared from these individual monomers include poly (D,L-lactide) (PLA), poly (glycolide) (PGA), and the copolymer poly (D,L-lactide-co-glycolide) (PLG).

Pharmaceutical compositions for oral administration may be prepared according to any method known to the art and may further comprise one or more agents selected from sweetening agents, flavoring agents, coloring agents and preserving agents in order to provide pharmaceutically elegant and palatable preparations. Tablets contain the active agents in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binding agents, e.g., starch, gelatin or acacia; and lubricating agents, e.g., magnesium stearate, stearic acid, or talc. The tablets may be either uncoated or coated utilizing known techniques to delay disintegration and absorption in the gastrointestinal tract and thereby provide a sustained action over a longer period. For example, a time delay material such as glyceryl monostearate or glyceryl distearate may be employed. They may also be coated using the techniques described in the U.S. Pat. Nos. 4,256,108, 4,166,452 and 4,265,874 to form osmotic therapeutic tablets for control release. The pharmaceutical composition of the invention may also be in the form of oil-in-water emulsion.

Useful dosage forms of the pharmaceutical compositions include orally disintegrating systems including, but not limited to, solid, semi-solid and liquid systems including disintegrating or dissolving tablets, soft or hard capsules, gels, fast dispersing dosage forms, controlled dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand-alone films and dry multiple layer stand-alone films.

The pharmaceutical composition of the invention may comprise one or more pharmaceutically acceptable excipients. For example, a tablet may comprise at least one filler, e.g., lactose, ethylcellulose, microcrystalline cellulose, silicified microcrystalline cellulose; at least one disintegrant, e.g., cross-linked polyvinylpyrrolidinone; at least one binder, e.g., polyvinylpyridone, hydroxypropylmethyl cellulose; at least one surfactant, e.g., sodium laurylsulfate; at least one glidant, e.g., colloidal silicon dioxide; and at least one lubricant, e.g., magnesium stearate.

Pharmaceutical compositions for rectal administration may be in any suitable form, e.g., a liquid or gel for injection into the lower bowel by way of the rectum using an enema, or formulated as a suppository, i.e., a solid dosage form for insertion into the rectum.

The pharmaceutical composition of the invention may be in the form of a sterile injectable aqueous or oleagenous suspension, which may be formulated according to the known art using suitable dispersing, wetting or suspending agents. The sterile injectable preparation may also be an injectable solution or suspension in a non-toxic parenterally acceptable diluent or solvent. Acceptable vehicles and solvents that may be employed include, without limiting, water, Ringer's solution, polyethylene glycol (PEG), 2-hydroxypropyl-β-cyclodextrin (HPCD), a surfactant such as Tween-80, and isotonic sodium chloride solution.

Pharmaceutical compositions according to the invention, when formulated for inhalation, may be in any suitable form, e.g., liquid or fine powder, and may be administered utilizing any suitable device known in the art, such as pressurized metered dose inhalers, liquid nebulizers, dry powder inhalers, sprayers, thermal vaporizers, electrohydrodynamic aerosolizers, and the like.

The pharmaceutical compositions of the present invention, as defined in any one of the embodiments above, are useful in preventing, inhibiting, reducing or ameliorating iron overload or elevated levels of labile iron, thereby more specifically treating a disease, disorder or condition characterized by or associated with iron overload or elevated levels of labile iron, as defined above.

The pharmaceutical compositions of the invention may be administered, e.g., continuously, daily, twice daily, thrice daily or four times daily, for various duration periods, e.g., weeks, months, years, or decades. The dosages will depend on the state of the patient, and will be determined, from time to time, as deemed appropriate by the practitioner. For example, a physician or veterinarian could start doses of the active agents employed in the pharmaceutical composition at levels lower than required in order to achieve the desired therapeutic effect, and gradually increase the dosage until the desired effect is achieved.

In still another aspect, the present invention relates to an iron chelator combination as defined above, i.e., a combination of a non-iron metal-DFO complex or a pharmaceutically acceptable salt thereof, and an additional iron chelator optionally partly or fully saturated with a non-redox active metal ion, for use in preventing, inhibiting, reducing, or ameliorating iron overload or elevated levels of labile iron.

In yet another aspect, the present invention relates to use of a combination of a non-iron metal-DFO complex or a pharmaceutically acceptable salt thereof, and an additional iron chelator optionally partly or fully saturated with a non-redox active metal ion in the preparation of a pharmaceutical composition for preventing, inhibiting, reducing, or ameliorating iron overload or elevated levels of labile iron.

As previously shown, DFO is capable of abstracting metals such as Fe and Zn from human plasma in vitro (Sooriyaarachchi and Gailer, 2010). It is thus postulated that under physiological conditions, administration of DFO or a pharmaceutically acceptable salt thereof, and metal ions, e.g., Zn- or Ga-ions, from two separate compositions, either concomitantly or sequentially (provided that the interval between administrations of the two components is determined such that at least a major amount of the component first administered is available in the circulation, i.e., not yet excreted, at the time the second component is administered), will result in the formation of a metal-DFO complex, or a pharmaceutically acceptable salt thereof, in situ.

The present invention thus further relates to a method for preventing, inhibiting, reducing or ameliorating iron overload or elevated levels of labile iron in a subject in need thereof, similar to the method defined above, wherein instead of administering a therapeutically effective amount of a non-iron metal-DFO complex or a pharmaceutically acceptable salt thereof, said subject is administered with amounts of (i) DFO or a pharmaceutically acceptable salt thereof; and (ii) ions of at least one metal other than iron, either concomitantly or sequentially at any order and within a time period not exceeding 36 hours, so as to form in situ upon complexation of said DFO or pharmaceutically acceptable salt thereof and said metal ions, a therapeutically effective amount of said non-iron metal-DFO complex or pharmaceutically acceptable salt thereof, which acts together with the additional iron chelator administered to prevent, inhibit, reduce or ameliorate iron overload or elevated levels of labile iron in said subject.

In certain embodiments, the DFO or pharmaceutically acceptable salt thereof, and the ions of the metal, are administered from two separate pharmaceutical compositions using the same or different administration modes, either concomitantly or sequentially at any order and within a time period not exceeding 36 hours, e.g., within a time period of up to about 2, 4, 6, 8, 10, 12, 14, 16, 18, 20, 22, 24, 26, 28, 30, 32, 34 or 36 hours, such that at least a major amount of the component first administered is available in the circulation at the time the second component is administered, and said metal-DFO complex or pharmaceutically acceptable salt thereof may thus be formed in situ.

Examples of metal ions and iron chelators that may be administered according to the method described hereinabove are listed above. In partiuclar embodiments, the metal ions administered are ions of Zn; Ga; or a nixture of Zn and any one of the other non-iron metal ions listed above, e.g., Ga, e.g., wherein the quantitative ratio of the Zn ions to the other non-iron metal ions is in a range of 100:1 to 1:100.

The metal ions administered may be in the form of cations (salts) in any possible valence state (depending on the specific metal), or in complexes with organic compounds such as aromatic and non-aromatic compounds having a heteroatom-containing moiety, e.g., carbonyl compounds, hydroxy compounds, heterocyclic compounds. Non-limiting examples of ligands (mono-, bi-, tridentate-, etc.) forming metal complexes are acetate, gluconate and acetylacetone, tris(2-aminoethyl)amine, crown ethers, porphyrins, alkyl phosphates such as dialkyldithiophosphate, and heterocycles such as terpyridine, pyrithione and metallocenes.

For example, zinc ions may be present in the form of a zinc salt, e.g., ZnCl₂ , or in complexes such as zinc acetate, zinc crown ether, Zn-porphyrin/crown ether conjugate, zinc protoporphyrin, zinc chlorophyll and bacteriochlorophyll, monomeric zinc dialkyldithiophosphate, zinc acetylacetone (trimer; Zn₃(AcAc)₆), zinc terpyridine (tridentate; [Zn(Terpy)Cl₂]), zinc tris(2-aminoethyl)amine, carbonic anhydrase (Zn metalloenzyme), glutamate carboxypeptidase II (Zn metalloenzyme), organozinc compounds such as diethylzinc (I) and decamethyldizincocene (II), Zinc gluconate, and zinc pyrithione. Gallium ions may be present in the form of a gallium salt, e.g., GaCl₃.

As defined above, the non-iron metal-DFO complex or pharmaceutically acceptable salt thereof formed in situ, and the additional iron chelator administered may be at any quantitative ratio, e.g., at a quantitative ratio in a range of 100:1 to 1:100 as defined above.

In a further aspect, the present invention provides a kit comprising (i) either a pharmaceutical composition A comprising a non-iron metal-DFO complex or a pharmaceutically acceptable salt thereof; or pharmaceutical compositions B and C, wherein pharmaceutical composition B comprises DFO or a pharmaceutically acceptable salt thereof, and pharmaceutical composition C comprises ions of a non-iron metal; (ii) a pharmaceutical composition D comprising an additional iron chelator optionally partly or fully saturated with a non-redox active metal ion; and (iii) instructions to administer either (a) pharmaceutical compositions A and D, either concomitantly or sequentially at any order and within a time period not exceeding 36 hours, e.g., within a time period of up to about 2, 4, 6, 8, 10, 12, 14, 16, 18, 20, 22, 24, 26, 28, 30, 32, 34 or 36 hours, to thereby prevent, inhibit, reduce, or ameliorate iron overload or elevated levels of labile iron, thus more particularly treat a disease, disorder or condition characterized by or associated with iron overload; or (b) pharmaceutical compositions B, C and D, either concomitantly or sequentially at any order and within a time period not exceeding 36 hours, so as to form in situ, upon complexation of said DFO or pharmaceutically acceptable salt thereof and said metal ions, a non-iron metal-DFO complex or a pharmaceutically acceptable salt thereof, to thereby prevent, inhibit, reduce, or ameliorate iron overload or elevated levels of labile iron, thus more particularly treat a disease, disorder or condition characterized by or associated with iron overload or elevated levels of labile iron, even under normal range of total body iron content.

In certain embodiments, the non-iron metal-DFO complex comprised within pharmaceutical composition A is the zinc-DFO complex, gallium-DFO complex, manganese-DFO complex, copper-DFO complex, aluminum-DFO complex, vanadium-DFO complex, indium-DFO complex, chromium-DFO complex, gold-DFO complex, silver-DFO complex, or platinum-DFO complex, a lanthanide-DFO complex, or a mixture thereof in any quantitative ratio. In other embodiments, the non-iron metal ions comprised within pharmaceutical composition C are ions of zinc, gallium, manganese, copper, aluminum, vanadium, indium, chromium, gold, silver, platinum, a lanthanide, or a nixture thereof in any quantitative ratio. The metal ions may be in the form of cations in any possible valence state, or in complexes with organic compounds such as aromatic and non-aromatic compounds having a heteroatom-containing moiety, as defined above.

In certain embodiments, the non-iron metal-DFO complex comprised within pharmaceutical composition A is Zn-DFO complex, Ga-DFO complex, or a mixture of Zn-DFO complex and any one of the other non-iron metal-DFO complexes listed above, e.g., Ga-DFO complex, e.g., wherein the quantitative ratio of said Zn-DFO complex to said other metal-DFO complex in said mixture is in a range of 100:1 to 1:100; or said pharmaceutical composition C comprises ions of Zn, Ga, or Zn and any one of the other metal ions listed above, e.g., Ga, e.g., wherein the quantitative ratio of the Zn ions to the other metal ions is in a range of 100:1 to 1:100.

In certain embodiments, the amount of the non-iron metal-DFO complex or pharmaceutically acceptable salt thereof comprised in pharmaceutical composition A, or alternatively, the amounts of the DFO complex or pharmaceutically acceptable salt thereof comprised in pharmaceutical composition B, and of the non-iron metal ions comprised in pharmaceutical composition C, are determined such that the metal-DFO complex administered or formed in situ, and the additional iron chelator, are at any quantitative ratio, e.g., at a quantitative ratio in a range of 100:1 to 1:100 as defined above.

The pharmaceutical compositions contained within the kit of the invention may be formulated, each independently, for any suitable administration route, as defined above.

The kit disclosed herein may comprise each one of the compositions in a ready for use form, e.g., formulated as a liquid for topical, nasal or oral administration, or may alternatively include one or both of the compositions as a solid composition that can be reconstituted with a solvent to provide a liquid oral dosage form. In cases one or more of the compositions are provided in a solid form for reconstitution with a solvent, the kit may further include a reconstituting solvent and instructions for dissolving said solid composition in said solvent prior to administration. Such a solvent should be pharmaceutically acceptable and may be, e.g., water, an aqueous liquid such as phosphate buffered saline (PBS), a non-aqueous liquid, or a combination of aqueous and non-aqueous liquids. Suitable non-aqueous liquids include, but are not limited to, oils, alcohols such as ethanol, glycerin, and glycols such as polyethylene glycol and propylene glycol.

The kit of the present invention, according to any one of the embodiments defined above, is useful for preventing, inhibiting, reducing, or ameliorating iron overload or elevated levels of labile iron, and thus for treating diseases, disorders, or conditions characterized by or associated with such medical conditions.

Unless otherwise indicated, all numbers expressing quantities of ingredients and so forth used in the present description and claims are to be understood as being modified in all instances by the term “about”. Accordingly, unless indicated to the contrary, the numerical parameters set forth in this specification and attached claims are approximations that may vary by up to plus or minus 10% depending upon the desired properties sought to be obtained by the present invention.

The invention will now be illustrated by the following non-limiting Examples.

### EXAMPLES

### Example 1. Treatment of Chronic Kidney Disease in a Murine Model

In this study the therapeutic effect of Zn-DFO and the zinc complex of the oral iron chelator deferasirox (Zn-DFX), administered together in combination, is tested on rats' CKD model (Naito et al., 2015). CKD is induced by 5/6 nephrectomy in Sprague-Dawley rats. At 8 weeks after operation, 5/6 nephrectomized rats are divided into 5 groups, as following: Group 1: untreated; Group 2: treated daily with deferasirox (DFX), 30 mg/kg/day, by oral gavage for 3 weeks; Group 3: treated daily with Zn-DFX, 30 mg/kg/day, by oral gavage for 3 weeks; Group 4: treated with Zn-DFO by i.p. injections, 6 mg/kg thrice a week for 3 weeks; Group 5: treated with Zn-DFX 10 mg/kg/day by oral gavage, together with Zn-DFO i.p. injections 4 mg/kg thrice a week for 3 weeks. Sham-treated animals serve as a control group. During 3 weeks after treatment systolic blood pressure, urinary protein secretion and serum creatinine are monitored. At 6 weeks after surgery, rats are euthanized, and kidneys are excised for semi-quantitative assessment of iron content and renal fibrosis.

Treatment either with DFX, Zn-DFX or Zn-DFO alone is expected to reduce urinary protein secretion and serum creatinine in CKD rats by 30%, while no effect on blood pressure is expected. Combined treatment with Zn-DFO and Zn-DFX, even at lower doses than when given separately, is expected to reduce urinary protein secretion and serum creatinine by about 80%, to slightly above the normal level. Furthermore, combined treatment is expected to reduce the systolic blood pressure from about 155 mm Hg to about 120 mm Hg, while the control animals will demonstrate the value of 100 mm Hg.

Histological assessment of renal fibrosis in these CKD model (as described in Naito et al., 2015) is expected to show reduction of severity score from 2.5 in the CKD group to 1.5 (or less) in each one of the Zn-DFO- and Zn-DFX-treated groups, and to 0.9 in the group treated with the combination of the two complexes. Renal iron content score is expected to be diminished from 2.7 in the CKD group to 1.8 in each one of the groups treated with either Zn-DFO or Zn-DFX, and to 1.5 in the group treated with the combination of the two complexes. Normal renal iron content score is expected to be 1.5.

### Example 2. Treatment of Chemotherapy-Induced Cardiomyopathy

The purpose of this study was to assess the protective effect of dexrazoxan or the zinc-dexrazoxan complex, administered with or without Ga-DFO, against doxorubicin-induced cardiomyopathy.

C57BL/6 mice are injected once with doxorubicin, 20 mg/kg, i.p., and then divided into the following 5 groups: Group 1: untreated; Group 2: treated with dexrazoxan single dose of 400 mg/kg i.v.; Group 3: treated with the zinc complex of dexrazoxan single dose of 100 mg/kg i.v.; Group 4: treated with the Ga-DFO complex 6 mg/kg, and Group 5: treated with the zinc complex of dexrazoxan single dose of 50 mg/kg i.v. together with Ga-DFO 4 mg/kg. The animals are monitored for 21 days, measuring their survival (Kaplan-Meier curves). On Day 21 the animals are sacrificed, and their hearts are assessed histologically for cardiac remodeling and hypertrophy (collagen % or area).

While about 90% of the doxorubicin-injected, but untreated mice are expected to die by Day 15 of the study, each one of the dexrazoxan alone or the zinc-dexrazoxan complex or Ga-DFO complex, is expected to improve the survival at this time point to about 50% without further degradation. Combined administration of the zinc-dexrazoxan complex with Ga-DFO is expected to improve the survival to about 65%.

In the untreated group collagen is expected to reach about 10-12% of the area, while each one of the dexrazoxan alone, zinc-dexrazoxan complex or Ga-DFO complex is expected to reduce this value to about 9%, about 6% and about 6%, respectively. Combined treatment with the zinc-dexrazoxan complex and Ga-DFO is expected to diminish it further to about 5%.

### REFERENCES

Bibi H., Vinokur V., Waisman D., Elenberg Y., Landesberg A., Faingersh A., Yadid M., Brod V., Pesin J., Berenshtein E., Eliashar R., Chevion M., Zn/Ga-DFO iron-chelating complex attenuates the inflammatory process in a mouse model of asthma. Redox Biol., 2014, 2, 814-819

Chevion M., A site-specific mechanism for free radical induced biological damage: the essential role of redox-active transition metals. Free Radic. Biol. Med., 1988, 5, 27-37

Chevion M., Protection against free radical-induced and transition metal-mediated damage: the use of “pull” and “push” mechanisms. Free Radic. Res. Commun., 1991, 12-13, 691-696

Gammella E., Maccarinelli F., Buratti P., Recalcati S., Cairo G., The role of iron in anthracycline cardiotoxicity. Front. Pharmacol., 2014, 5, 25

Hider R. C., Charge states of deferasirox-ferric iron complexes. Am. J. Kidney Dis., 2010, 55, 614-615

Keberle H., The biochemistry of desferrioxamine and its relation to iron metabolism. Ann. NY Acad. Sci., 1964, 119, 758-768

Kornreich-Leshem H., Ph.D. Thesis “Biomimetic siderophore derivatives as specific iron chelators; 2003; The Weizmann Institute of Science, Israel

Kornreich-Leshem H., Ziv C., Gumienna-Kontecka E., Arad-Yellin R., Chen Y., Elhabiri M., Albrecht-Gary A. M., Hadar Y., Shanzer A., Ferrioxamine B analogues: Targeting the FoxA uptake system in the pathogenic yersinia enterocolitica. J. Am. Chem. Soc., 2005, 127(4), 1137-1145

Naito Y., Fujii A., Sawada H., Oboshi M., Iwasaku T., Okuhara Y., Morisawa D., Eguchi A., Hirotani S., Masuyama T., Association between renal iron accumulation and renal interstitial fibrosis in a rat model of chronic kidney disease. Hypertens. Res. 2015, 38, 463-470

Orcutt K. M., Jones W. S., McDonald A., Schrock D., Wallace K. J., A lanthanide-based chemosensor for bioavailable Fe3+ using a fluorescent siderophore: an assay displacement approach. Sensors (Basel), 2010, 10, 1326-1337

Origa R, Bina P, Agus A, Crobu, G., Defraia, E., Dessi, C., Leoni G., Muroni P. P., Galanello, R., Combined therapy with deferiprone and desferrioxamine in thalassemia major. Haematologica, 2005, 90, 1309-1314

Sooriyaarachchi M., Gailer J., Removal of Fe³⁺ and Zn²⁺ from plasma metalloproteins by iron chelating therapeutics depicted with SEC-ICP-AES. Dalton Trans., 2010, 39, 7466-7473

1. A pharmaceutical composition comprising a combination of a metal-desferrioxamine B complex (metal-DFO complex) or a pharmaceutically acceptable salt thereof, wherein said metal is not iron, and an additional iron chelator, and a pharmaceutically acceptable carrier.

2. The pharmaceutical composition of claim 1, wherein said metal-DFO complex is the zinc-, gallium-, manganese-, copper-, aluminum-, vanadium-, indium-, chromium-, gold-, silver- or platinum-DFO complex, a lanthanide-DFO complex, or a mixture thereof.

3. The pharmaceutical composition of claim 2, wherein said metal-DFO complex is Zn-DFO complex, Ga-DFO complex, or a mixture thereof.

4. The pharmaceutical composition of claim 3, wherein said metal-DFO complex is a mixture of Zn-DFO complex and Ga-DFO complex, and the quantitative ratio of said Zn-DFO complex to said Ga-DFO complex in said mixture is in a range of 100:1 to 1:100.

5. The pharmaceutical composition of claim 1, wherein said additional iron chelator is a natural siderophore such as desferrioxamine D, and desferrioxamine E (nocardamine); a chemically modified siderophore such as a desferrioxamine B analog; desferrithiocin; kojic acid; dihydroxybenzoic acid; a synthetic chelator such as deferiprone, ethylene-diamine-tetra-acetic acid, clioquinol, dimercaptosuccinic acid, O-trensox, deferasirox, dexrazoxan, tachpyr, triapine, dimercaprol, penicillamine, pyridoxal isonicotinoyl hydrazone, and di-2-pyridylketone thiosemicarbazone; a phytochemicalsuch as genistein, phytic acid, theaflavin, epigallochatechin gallate, quercetin, ligustrazine, baicalin, baicalein, floranol, kolaviron, apocynin, flavan-3-ol, and curcumin; or a pharmaceutically acceptable salt thereof.

6. The pharmaceutical composition of claim 5, wherein said additional iron chelator is in a metal-free form.

7. The pharmaceutical composition of claim 5, wherein said additional iron chelator is in a complex with zinc, gallium, manganese, copper, aluminum, vanadium, indium, chromium, gold, silver, platinum, a lanthanide, or a mixture thereof.

8. The pharmaceutical composition of claim 1, wherein the quantitative ratio of said metal-DFO complex to said additional iron chelator in said combination is in a range of 100:1 to 1:100.

9. The pharmaceutical composition of claim 1, formulated for oral, sublingual, buccal, rectal, intravenous, intraarterial, intramuscular, intraperitoneal, intrathecal, intrapleural, intratracheal, cutaneous, subcutaneous, transdermal, intradermal, nasal, vaginal, ocular, otic, or topical administration, or for inhalation.

10. The pharmaceutical composition of any one of claims 1 to 9, for preventing, inhibiting, reducing or ameliorating iron overload or elevated levels of labile iron.

11. The pharmaceutical composition of claim 10, for treatment of pantothenate kinase-associated neurodegeneration (PKAN), human immunodeficiency virus infection and acquired immune deficiency syndrome (HIV/AIDS), intracerebral hemorrhage, myelodysplastic syndrome, Hodgkin lymphoma, non-Hodgkin lymphoma, hepatic insufficiency, renal failure, sickle-cell disease, Parkinson's disease, Friedreich's ataxia, thalassemia, amyotrophic lateral sclerosis (ALS), neurodegeneration with brain iron accumulation (NBIA), superficial siderosis, contrast-induced acute kidney injury (CI-AKI), iron overload due to stem cell transplant, mucormycosis, acute myeloid leukemia (ACM), Diamond-Blackfan anemia, hemolytic anemia, porphyria cutanea tarda, malaria, acute lymphoid leukemia, hemosiderosis, non-alcoholic steatohepatitis, aplastic anemia, diabetic nephropathy, glomerulonephritis, rheumatoid arthritis, endotoxemia, stroke, chronic kidney disease (CKD), systemic sclerosis, Wilson's disease, Menkes disease, glioblastoma, pulmonary fibrosis, idiopathic pulmonary fibrosis, chronic hemophilic synovitis, Alzheimer's disease, Huntington's disease, schizophrenia, cystinuria, biliary cirrhosis, leishmaniasis, multiple sclerosis, cholangiocarcinoma, primary sclerosing cholangitis (PSC), heavy metals poisoning, autoimmune encephalomyelitis, carcinoma, fibrosarcoma, fibroma, histiocytoma, myxosarcoma, angiomyxoma, adenoma, mesothelioma, hepatoblastoma, adenocarcinoma, cholangiocarcinoma, cystadenoma, melanoma, sarcoma, hemangioma, teratoma, adenomyoma, leiomyosarcoma, oncocytoma, inverted papilloma, papilloma, chemotherapy-induced iron overload, inflammatory bowel disease, ulcerative colitis, Crohn's disease, diabetes mellitus, metabolic syndrome, or psoriasis.

12. The pharmaceutical composition of claim 10, for treatment of a disorder or condition induced by an injury, such as an injury caused by a mechanical force, ischemia, a toxic agent such as an herbicide or pesticide, or hemorrhage.

13. A combination of a metal-desferrioxamine B complex (metal-DFO complex) or a pharmaceutically acceptable salt thereof, wherein said metal is not iron, and an additional iron chelator for use in preventing, inhibiting, reducing, or ameliorating iron overload or elevated levels of labile iron.

14. Use of a combination of a metal-desferrioxamine B complex (metal-DFO complex) or a pharmaceutically acceptable salt thereof, wherein said metal is not iron, and an additional iron chelator in the preparation of a pharmaceutical composition for preventing, inhibiting, reducing, or ameliorating iron overload or elevated levels of labile iron.

15. A kit comprising:
(i) either a pharmaceutical composition A comprising a metal-desferrioxamine B complex (metal-DFO complex) or a pharmaceutically acceptable salt thereof; or pharmaceutical compositions B and C, wherein pharmaceutical composition B comprises DFO or a pharmaceutically acceptable salt thereof, and pharmaceutical composition C comprises ions of a metal, wherein said metal is not iron;
(ii) a pharmaceutical composition D comprising an additional iron chelator; and
(iii) instructions to administer either (a) pharmaceutical compositions A and D, either concomitantly or sequentially at any order and within a time period not exceeding 36 hours; or (b) pharmaceutical compositions B, C and D, either concomitantly or sequentially at any order and within a time period not exceeding 36 hours, so as to form in situ, upon complexation of said DFO or pharmaceutically acceptable salt thereof and said metal ions, a metal-DFO complex or a pharmaceutically acceptable salt thereof,
to thereby prevent, inhibit, reduce, or ameliorate iron overload or elevated levels of labile iron.

16. The kit of claim 15, wherein said metal-DFO complex is the zinc-, gallium-, manganese-, copper-, aluminum-, vanadium-, indium-, chromium-, gold-, silver- or platinum-DFO complex, a lanthanide-DFO complex, or a combination thereof; or said pharmaceutical composition C comprises ions of zinc, gallium, manganese, copper, aluminum, vanadium, indium, chromium, gold, silver, platinum, a lanthanide, or a mixture thereof.

17. The kit of claim 15, wherein said metal-DFO complex is Zn-DFO complex, Ga-DFO complex, or a mixture thereof; or said pharmaceutical composition C comprises ions of Zn, Ga, or both Zn and Ga.

18. The kit of claim 17, wherein said metal-DFO complex is a mixture of Zn-DFO complex and Ga-DFO complex, and the quantitative ratio of said Zn-DFO complex to said Ga-DFO complex in said mixture is in a range of 100:1 to 1:100; or said pharmaceutical composition C comprises ions of both Zn and Ga, and the quantitative ratio of the Zn ions to the Ga ions is in a range of 100:1 to 1:100.

19. The kit of claim 15, wherein said additional iron chelator is a natural siderophore such as desferrioxamine D, and desferrioxamine E (nocardamine); a chemically modified siderophore such as a desferrioxamine B analog; desferrithiocin; kojic acid; dihydroxybenzoic acid; a synthetic chelator such as deferiprone, ethylene-diamine-tetra-acetic acid, clioquinol, dimercaptosuccinic acid, O-trensox, deferasirox, dexrazoxan, tachpyr, triapine, dimercaprol, penicillamine, pyridoxal isonicotinoyl hydrazone, and di-2-pyridylketone thiosemicarbazone; or a phytochemicalsuch as genistein, phytic acid, theaflavin, epigallochatechin gallate, quercetin, ligustrazine, baicalin, baicalein, floranol, kolaviron, apocynin, flavan-3-ol, and curcumin; or a pharmaceutically acceptable salt thereof.

20. The kit of claim 19, wherein said additional iron chelator is in a metal-free form.

21. The kit of claim 19, wherein said additional iron chelator is in a complex with zinc, gallium, manganese, copper, aluminum, vanadium, indium, chromium, gold, silver, platinum, a lanthanide, or a mixture thereof.
